(19) United States
(12) Patent Application Publication
(10) Pub. No.: US 2013/0344486 A1
(43) Pub. Date: Dec. 26, 2013

(54) METHOD OF DIAGNOSING BREAST CARCINOMA

(71) Applicants: Manuel Debald, Bonn (DE); Gisela Walgenbach-brunagel, Bonn (DE)

(72) Inventors: Manuel Debald, Bonn (DE); Gisela Walgenbach-brunagel, Bonn (DE)

(73) Assignee: Rheinische Friedrich-Wilhelms-Universitat Bonn, Bonn (DE)

(21) Appl. No.: 14/001,900

(22) PCT Filed: Feb. 28, 2012
(86) PCT No.: PCT/EP2012/053349

§ 371 Date: Aug. 28, 2013

(30) Foreign Application Priority Data
Feb. 28, 2011 (EP) 11156242.7

(51) Int. Cl. G01N 33/68

(52) U.S. Cl. CPC G01N 33/6893
USPC 435/6.11; 436/501; 435/6.14; 435/7.23; 435/7.1; 435/7.92; 435/6.12

(57) ABSTRACT

The present invention relates to methods of diagnosing a breast carcinoma, determining the prognosis of a patient diagnosed with breast carcinoma and determining the efficacy of a treatment regimen of breast carcinoma in a patient, using Calponin-h2 and/or CALML 5 as markers. Furthermore, the invention relates to a kit and a marker panel for use in these methods.

### FIELD OF THE INVENTION

The present invention relates to methods of diagnosing abreast carcinoma, determining the prognosis of a patient diagnosed with breast carcinoma and determining the efficacy of a treatment regimen of breast carcinoma in a patient, using Calponin-h2 and/or CALML 5 as markers. Furthermore, the invention relates to a kit and a marker panel for use in these methods.

### BACKGROUND

Breast cancer is the most common cancer in women. Every year more than 1.3 million women worldwide are diagnosed with breast cancer, whereby nearly 500.000 patients die due to the fatal course of this disease. The early diagnosis of breast cancer in a potentially curable stage improves the prognosis and consecutively reduces mortality of breast cancer diagnosed patients. Currently, clinical breast examination, imaging by mammography as well as tumor biopsy are the only methods recommended for breast cancer screening in the non-high risk population. The established screening by breast examination and mammography is able to detect breast cancer in early stages and has been shown to reduce mortality of patients diagnosed for breast cancer. Nevertheless, screening by mammography, especially in patients younger than 50 years, remains controversial. This is in particular due to significant rates of false negative as well as false positive results leading to overdiagnosis and overtherapy. Importantly, young women (<50 years) with a high density of the breast show low sensitivities in mammography. The sensitivity of mammography in women aged 50 years and older ranges from 68% to 90%, in women aged 40-49 the sensitivity is lower, with an estimate of between 62% and 76%. Likewise, a meta-analysis of randomized controlled trials showed a decreased mortality reduction of 15% in young women (39-49 years) undergoing regular screening by mammography, compared to 30% in women aged 49-59. Therefore, established mammography screening programs are mainly addressed to older women (50-69 years). However, breast cancer is an especially important issue for the non-screened women belonging to the younger subgroup, as among this population one fifth of new breast cancers cases occurs, which are in this subgroup often aggressive and in a fast growing form. Moreover, breast cancer is the most common cause of cancer death among young females aged 20-59 years. For this reason, there is a special need for a test which allows reliable diagnosis of breast cancer in young women. In addition, mammography usually requires the patient to make an appointment with a mammography center for analysis. These centers are rare and thus the patient is usually required to travel a significant distance to the center. For this reason about half of patients leave out an institutional invitation to a screening. Accordingly, there is a demand for a diagnostic method which can be employed by any physician and that avoids inconvenience for the patients. In addition, the slots for the breast cancer diagnostic test are rare in screening centers and thus appointments are seldom given to the patient. A frequent control of the patient is particularly important if the treatment of a breast cancer patient is to be monitored. Hence, there is need for a breast cancer diagnostic test which allows frequent analysis of patients. Also for economic reasons, as mammography is a high priced technology, there is a demand for a more cost effective breast cancer test.

During the last two decades, genomic and proteomic technologies have significantly increased the number of potential DNA, A and protein biomarkers in breast cancer. But so far, there is no reliable non-invasive test available for the clinical routine. Regarding proteomic approaches, a major challenge lies in the complexity of the human proteome and its dynamic state. Even current Mass Spectrometry (MS) based technologies are still failing to achieve large access to low abundant proteins in complex biological samples. However, low abundant proteins represent numerous potential tumor specific biomarkers.

Nuclear matrix proteins (NMPs) represent only 1% of the total cell proteome. In 1974, the nuclear matrix has been first described as the structural framework scaffolding of the nucleus, consisting of the peripheral lamins, protein complexes, an internal ribonucleic protein network and residual nucleoli. Most of the nuclear matrix proteins (NMPs) are common to all cell types, but numerous NMPs are tissue and cell type specific. The main characteristics of cancer cells are alterations in the size and shape of the nucleus that reflect the analogous alteration of the nuclear matrix. Recently, alterations of several NMP have been shown to be cancer specific biomarkers (Leman et al., J Cell Biochem, 104(6): 1988-1993, 2008). These findings have been successfully developed into non-invasive, blood- and urine-based test with high sensitivity and specificity for prostate, bladder as well as colon cancer, which have now to be validated in correctly designed large studies to demonstrate their clinical utility (Van Le et al., Urology, 66(6): 1256-1260, 2005; Leman et al., Cancer Res, 67(12): 5600-5605, 2007; Leman et al., Urology, 69(4): 714-720; 2007; Walgenbach-Brünagel et al., J Cell Biochem, 104(1): 286-294, 2008).

However, for none of the above detailed tests suitability as a breast cancer diagnostic test has been demonstrated. Accordingly, there is still need in the field for a reliable, preferably non-invasive, rapid diagnostic test which allows diagnosis of breast cancer.

### SUMMARY OF THE INVENTION

The present invention relates to a method of diagnosing breast carcinoma in a patient, wherein the method comprises determining the level of Calponin-h2 and/or CALML5 in a sample obtained from the patient, wherein if the level of Calponin-h2 and/or CALML5 is increased said patient is diagnosed with breast carcinoma.

In a further aspect, the present invention relates to a method of determining the prognosis of a patient diagnosed with breast carcinoma, wherein the method comprises determining the level of Calponin-h2 and/or CALML5 in a sample obtained from the patient, wherein, if the level of Calponin-h2 and/or CALML5 is increased, said patient has an increased likelihood of an adverse outcome.

The present invention is further directed to a method of determining the efficacy of a treatment regimen of breast carcinoma in a patient, wherein the method comprises determining the level of Calponin-h2 and/or CALML5 in a first sample obtained from the patient before said treatment regimen has commenced and a second sample obtained from the patient during or after said treatment regimen, wherein a decrease of the level of Calponin-h2 and/or CALML5 in the second sample relative to the first sample indicates that the treatment is effective.

In certain embodiments of any one of the methods described herein, the method comprises determining the level of one or more additional markers.

In various embodiments of any one of the methods described herein, the method comprises determining the level of one or more additional markers wherein the one or more additional markers are selected from the group consisting of hsp27, estrogen receptor, progesterone receptor, EGFR, Her2, circulating DNA, circulating RNA, circulating tumor cells, upa/PAI1, miRNA, ki67, Bone Sialoprotein, CA15-3, CA27.29, CEA, P53, Cathepsin D, Cyclin E, Vitronectin, Vimentin, S100, MMP11, CTSL2, STK15, Survivin, Cyclin B1, MYBL2, GSTM1, BAG1, ITIH4, C3a-complement, GCDFB-15, ApoD, alpha-1-acid glycoprotein, the protein in Spot C of the 2D-gel-electrophoresis (FIG. 1, 25 kDa, pI 4), the protein in Spot E of the 2D-gel-electrophoresis (FIG. 1, 20 kDa, pI 4,5), CA15-3, CA27.29, CEA, the protein in spot F of the 2D-gel-electrophoresis (FIG. 1, 20 kDa, pI 4), CEACAM1, tissue inhibitors of metalloproteinases (TIMP)-3, Calgranulin A, LyGDI, RhoA, Profilin, and Apo-C1.

In some embodiments of any one of the methods described herein, the sample is a biological sample.

In certain embodiments of any one of the methods described herein, the sample is a biological sample, wherein the biological sample is a body fluid, cell or tissue sample.

In various embodiments of any one of the methods described herein, the sample is a body fluid selected from the group consisting of blood, serum, plasma, urine, nipple aspirate fluid and saliva.

In certain embodiments of any one of the methods described herein, the patient is a human.

In some embodiments of any one of the methods described herein, determining the level of Calponin-h2 and/or CALML5 comprises determining the expression level of Calponin-h2 and/or CALML5.

In various embodiments of any one of the methods described herein, determining the level of Calponin-h2 and/or CALML5 comprises determining the Calponin-h2 and/or CALML5 protein and/or mRNA level in a sample.

In certain embodiments of any one of the methods described herein, determining the level of Calponin-h2 and/or CALML5 comprises determining the Calponin-h2 and/or CALML5 protein level in a sample, wherein the protein level is determined by an immunoassay, ELISA, mass spectrometry, chromatography, Western Blot, or gel electrophoresis.

In various embodiments of any one of the methods described herein, determining the level of Calponin-h2 and/or CALML5 comprises determining the Calponin-h2 and/or CALML5 mRNA level in a sample, wherein the mRNA level is determined by PCR, gel electrophoresis, or Northern Blot.

In a fourth aspect, the present invention concerns a kit for use in the methods of the invention, wherein the kit comprises reagents for determining the level of Calponin-h2 and/or CALML5 in a sample.

Moreover, the present invention relates to a marker panel comprising Calponin-h2 and CALML5 for use in a method of the invention.

### BRIEF DESCRIPTION OF THE DRAWINGS

FIG. 1 shows a representative gel of nuclear matrix proteins (100 μg) in human breast cancer after high-resolution two-dimensional gel electrophoresis and silver staining. Protein spots A-E, characteristic for breast cancer, are labeled. Also the estimated the molecular weights and pI are indicated.

FIG. 2 shows silver stainings after high-resolution two-dimensional gel electrophoresis of nuclear matrix proteins samples isolated from human breast cancer tissue (n=14, tumor 1-14), healthy control tissues (n=2, control 1 and 2), benign control tissues (n=2, control 3 and 4), a MCF10A cell line, which represents human normal epithelial breast cells, and human breast cancer cell lines Bt474 and HCC1937. It is apparent that protein spots A (left arrow) and B (right arrow) are present in human breast cancer tissue, but not expressed in human benign and healthy controls. Furthermore, these spots were also not detected in the MCF10A cell line, but present in the samples of the human breast cancer cell lines Bt474 and HCC1937.

FIG. 3 shows silver stainings after high-resolution two-dimensional gel electrophoresis of nuclear matrix proteins samples isolated from human breast cancer tissue (n=14, tumor 1-14), healthy control tissues (n=2, control 1 and 2), benign control tissues (n=2, control 3 and 4), a MCF10A cell line, which represents human normal epithelial breast cells, and human breast cancer cell line HCC1937. It is apparent that protein spot C is specific for human breast cancer, as it was not detected in human benign and healthy controls. Furthermore, this spot was also not detected in the MCF10A cell line but present in the human breast cancer cell line HCC1937.

FIG. 4 shows silver stainings after high-resolution two-dimensional gel electrophoresis of nuclear matrix proteins samples isolated from human breast cancer tissue (n=14, tumor 1-14), healthy control tissues (n=2, control 1 and 2), benign control tissues (n=2, control 3 and 4), a MCF10A cell line, which represents human normal epithelial breast cells, and human breast cancer cell line Bt474. It is apparent that protein spot D was specific for human breast cancer tissue as it was not detected in human benign and healthy controls. Furthermore, this spot was also not detected in the MCF10A cell line but present in the human breast cancer cell line Bt474.

FIG. 5 shows silver stainings after high-resolution two-dimensional gel electrophoresis of nuclear matrix proteins samples isolated from human breast cancer tissue (n=14, tumor 1-14), healthy control tissues (n=2, control 1 and 2), benign control tissues (n=2, control 3 and 4), a MCF10A cell line, which represents human normal epithelial breast cells, and human breast cancer cell lines Bt474, SkBr3 and HCC1937. It is apparent that protein spot E was specific for human breast cancer tissue as it was not detected in human benign and healthy controls. Furthermore, this spot was also not detected in the MCF10A cell line, but present in the human breast cancer cell lines Bt474, SkBr3 and HCC1937.

FIG. 6 shows Western-Blots of Nuclear matrix protein (NMP) extracts (10μ/lane) of ductal-invasive (Tumor 15), lobular-invasive (Tumor 16) and mucinous-invasive (Tumor 17) human breast cancers and of 2 healthy control tissues, and cell lysates of control cells H358 and HepG2, which were fractionated by SDS-PAGE. The total cell lysate of 1-1358 cells (human lung cancer cell line) served as a negative, the cytoplasmic lysates of HepG2 cells (human hepatocellular carcinoma cell line) as positive control. After electrophoresis the nitrocellulose Blot membrane was subjected to immunodetection with a specific antibody against human Calponin-h2, α-Tubulin (55 kDa) or Lamin A-C (70 kDa). A cytoplasmic contamination in the NMP samples could be excluded by a negative reaction for α-Tubulin. Lamin A-C was used as a loading control for the NMP fraction. A specific band at 37 kDa indicates the expression of Calponin h2 in the different human breast cancer subtypes. This band is not found in both human healthy breast tissue controls, indicating the specific expression of Calponin-h2 in human breast cancer.

FIG. 7 shows Western-Blots of Nuclear matrix protein (NMP) extracts, nuclear (nuc) and cytoplamic (cyto) protein extraxts (10μ/lane) of the breast cancer cell lines SkBr3 and Bt474, which were fractionated by SDS-PAGE. The nitrocellulose membrane was subjected to immunodetection with a specific antibody against Calponin-h2, α-Tubulin (55 kDa) or Lamin A-C (70 kDa). A cytoplasmic contamination of the investigated NMP extracts could be excluded by a negative reaction for α-Tubulin. The presence of a nuclear protein fraction was confirmed by a positive reaction for Lamin A-C. A specific band at 37 kDa indicates the nuclear expression of Calponin-h2 in the investigated breast cancer cell lines, confirming the epithelial source of Calponin-h2.

FIG. 8 shows Western-Blots of a cytoplasmic extract (10 μg/lane) of human breast cancer tissue (Tumor 15) which was fractionated by SDS-PAGE. The nitrocellulose membrane was to immunodetection with a specific antibody against Calponin-h2, α-Tubulin (55 kDa) or Lamin A-C (70 kDa). The specific antibody against human Calponin h2 did not detect Calponin h2 in the cytoplasmic fraction of human breast cancer tissue. The presence of cytoplasmic proteins was confirmed by a positive reaction for α-Tubulin (55 kDa). The absence of nuclear proteins was confirmed by a negative reaction for Lamin A-C (70 kDa). Hence, the data demonstrates that Calponin-h2 is not present in the cytoplasm of human breast cancer tissue.

FIG. 9 shows the Calponin-h2 serum levels (ng/ml) of early breast cancer patients and healthy controls, which were determined using Calponin-h2 ELISA.

FIG. 10 shows the results of a statistical analysis of the results shown in FIG. 9 demonstrating a significant difference in Calponin-h2 serum levels between early breast cancer patients and normal control women.

FIG. 11 shows the receiver operating characteristic (ROC) curve for Calponin-h2 in human serum: Early breast cancer versus healthy control patients. Generally, the accuracy of a test is described by its receiver—operating characteristics (ROC). The ROC graph is a plot of all of the sensitivity/specificity pairs resulting over the entire range of data observed. The y-axis represents the sensitivity (%), whereas the x-axis represents the 100%-specificity (%).

### DETAILED DESCRIPTION

The invention is based on the inventors' surprising finding that Calponin-h2 and/or CALML 5 are detectable in breast cancer tissue whereas both are not detected in healthy and benign control tissue and thus can serve as markers for breast carcinoma.

The present invention thus relates to methods of diagnosing breast carcinoma, determining the prognosis of a patient diagnosed with breast carcinoma and determining the efficacy of a treatment regimen of a patient with breast carcinoma, using Calponin-h2 and/or CALML 5 as markers for (i) the presence of breast carcinoma, (ii) an unfavorable prognosis of a patient with breast carcinoma, or (iii) efficacy of a treatment of breast carcinoma.

These markers can be detected in tissue and/or body fluid samples, e.g., in a blood sample, and thus provide for a novel method for the diagnosis of breast cancer. As such a method does not require expensive equipment, the costs for breast cancer diagnosis can be reduced. Furthermore, the new method can be carried out by any physician and therefore do not require the patient to travel to screening centers. This allows more frequent medical examinations. As mentioned in the introductory part, for patients with an age of below 50 years, mammography does not provide readout for a reliable breast cancer diagnosis. However, the present invention provides suitable methods for breast cancer detection in patients of any age group. As particularly young women suffer from severe forms of breast cancer, this is a significant advantage of the methods of the present invention.

Calmodulin-like protein 5 (CALML5=CLSP, SEQ ID No. 1, Uniprot Database entry: Q9NZT1, Feb. 8, 2011, Version 88) was discovered and biochemically characterized in 2000 by Mehul et al. (Mehul et al., J Biol Chem, 275(17): 12841-12847, 2000). As the protein binds calcium, has homology (52%) with CaM and a similar epidermal tissue distribution, it was named calmodulin-like skin protein (CLSP). The terms “CLSP” and “CALML5” are used interchangeably for this protein. Although it has been reported that in human breast cancer tissue gene expression patterns of CALML5 were found to be upregulated in invasive and metastatic breast cancer (Porter et al., Mol Cancer Res, 1(5): 362-375, 2003, CALML5 has not been suggested as breast cancer marker so far and the presence of CALML5 in human breast carcinoma on a protein level has not yet been described.

Furthermore, the inventors have identified Calponin-h2 (SEQ ID No. 2, Uniprot Database entry: Q99439, Feb. 8, 2011. Version 108), a member of the Calponin family, as a marker for breast carcinoma. Calponins are a family of 34-37 kDa cytoplasmic Ca2+-binding proteins, which bind in vitro to F-actin and tropomyosin (Takahashi et al., Biochem Biophys Res Commun, 141(1): 20-26, 1986). There are three isoforms (basic h1, neutral h2 and acidic h3) sharing high sequence identity within the first 273 amino acids, but differing significantly in their carboxy-terminal sequences (Tang et al., J Biol Chem, 281(10): 6664-6672, 2006). In 1993, Calponin-h2 has been identified by Strasser et al. which demonstrated that Calponin-h2 is encoded by a different gene than Calponin-h1 and -h3 (Strasser et al., FEBS Lett, 330(1): 13-18, 1993). This neutral isoform is expressed in both, smooth muscle as well as non-muscle cells.

It is found in fibroblasts and epidermal keratinocytes with a role in stabilizing the actin filaments. It is known, that calponins are involved in numerous functions in muscle and non-muscles cells. However, so far the expression of calponins, particularly of Calponin-h2, in cancer cells and their biological function has not been investigated.

In addition, the inventors have identified heat shock protein beta-1 (hsp27, SEQ ID No. 3, Uniprot Database entry: PO4792, Feb. 8, 2011. Version 141) as a suitable additional marker for the diagnosis of breast carcinoma. Overexpression of hsp27 has observed in numerous cancer entities and, in particular, in human breast cancer (Ciocca et al., Cell Stress Chaperones, 10(2): 86-103, 2005). Up-regulated protein levels have been associated with estrogen receptor levels as well as better differentiation of tumor cells suggesting a good prognosis. However, these data are still under debate as additional studies could not confirm these findings (Ciocca et al., Cell Stress Chaperones, 10(2): 86-103, 2005). Moreover, further studies indicated a link between high levels of hsp27 and more aggressive tumors as well as drug resistance (Oesterreich et al., Cancer Res, 53(19): 4443-4448, 1993; Hansen et al, Breast Cancer Res Treat, 56(2): 187-196, 1999). Likewise, hsp27 positive breast cancer from node-negative patients is correlated to lower overall survival and survival after first recurrence (Thanner et al., Anticancer Res, 25(3A): 1649-1653, 2005). The data show that heat shock proteins are involved in several aspects of tumor biology, but their definite role in cancer diagnosis, prognosis and therapy has not been clarified (Romanucci et al., Cell Stress Chaperones, 13(3): 253-262, 2008). Posttranslational modifications, especially phosphorylation of hsp27 (Ser78 and Ser82) have been shown to be associated with a nuclear translocation in transfection experiments (Geum et al., J Biol Chem, 277(22): 19913-19921, 2002). The inventors have demonstrated an expression of hsp27 in the nucleus of human breast cancer tissue. Without wishing to be bond to a specific theory, a posttranslational modification of nuclear hsp27 might explain the nuclear localization observed by the inventors. These modifications may be specific for human breast cancer tissue.

The inventors have found that the nuclear localization of Calponin-h2, CALML5 and hsp27 is specific for human breast cancer tissue compared to healthy and benign controls. Studies performed by the groups of Mann et al. and van Eyk et al. demonstrated the existence of Calponin-h2 as well as CALML5 in the human plasma proteome (Sheng et al., Mol Cell Proteomics, 5(1): 26-34, 2006; Schenk et al., BMC Med Genomics, 1: 41, 2008). As these proteins can be found in human blood, the inventors developed a blood based assay detecting the up-regulation of Calponin-h2 as well as CALML5 that can be used to separate healthy controls from breast cancer patients. Consequently, Calponin-h2 and/or CALML5 have been found to be useful as diagnostic and prognostic biomarkers in human breast carcinoma and allow improving the management of this disease.

In a first aspect, the present invention relates to a method of diagnosing breast carcinoma in a patient, wherein the method comprises determining the level of Calponin-h2 and/or CALML5 in a sample obtained from the patient, wherein if the level of Calponin-h2 and/or CALML5 is increased said patient is diagnosed with breast carcinoma.

In a second aspect, the present invention relates to a method of determining the prognosis of a patient diagnosed with breast carcinoma, wherein the method comprises determining the level of Calponin-h2 and/or CALML5 in a sample obtained from the patient; wherein, if the level of Calponin-h2 and/or CALML5 is increased, said patient has an increased likelihood of an adverse outcome.

In a third aspect, the present invention relates to a method of determining the efficacy of a treatment regimen of breast carcinoma in a patient, wherein the method comprises determining the level of Calponin-h2 and/or CALML5 in a first sample obtained from the patient before said treatment regimen has commenced and a second sample obtained from the patient during or after said treatment regimen; wherein a decrease of the level of Calponin-h2 and/or CALML5 in the second sample relative to the first sample indicates that the treatment is effective.

An increased level of a marker means that its concentration is increased relative to a normal state, i.e. a healthy individual not afflicted by breast carcinoma. This term includes that in the normal healthy state the marker is not detectable, e.g. is present in levels below the detection limit, but can be detected in breast carcinoma patients. It is also possible to define a threshold level, where when the determined level is above this level, it is defined as increased.

Determining the prognosis includes risk stratification and prediction of the likelihood of an adverse outcome. This can be made in relation to a certain time period. Adverse outcome in the sense of the present invention include deterioration of a patient's condition, for example due to metastasis, and also death.

Increased likelihood means that compared to an individual where the marker levels are not increased, the chance that a certain event occurs is higher. The increase may be 5%. The treatment regimen the efficacy of which is monitored is usually an anti-cancer therapy. This may include chemotherapy, but also therapy by radiation or surgery. Anti-cancer medicaments that can be used for the treatment of breast carcinoma are well known to those skilled in the art. The determination of the efficacy of the treatment regimen usually involves comparing marker levels before and during or after said treatment. However, this method may also include determining a first level at an early stage of treatment and a second level at a later stage of treatment. It is also included that marker levels are determined at additional time points. Accordingly, the method may comprise determining markers levels daily, every two days, weekly or monthly over a certain period of time, for example one, two, three or more months or even a year or more.

Calponin-h2 and CALML5 are preferably human Calponin-h2 and CALML5.

In various embodiments, the methods of the invention can further comprise determining the level of one or more additional markers. The additional marker may, for example, be hsp27. In further embodiments, the methods may comprise the determination of hsp27 levels and at least one or more further markers. In certain embodiments the one or more additional markers can be selected from the group consisting of nuclear matrix proteins (NMPs) or other makers. The phrase “nuclear matrix” refers to a 3-dimensional filamentous protein network that is present in the interphase nucleus. The NMPs of the protein network provide a framework to maintain the overall size and shape of the nucleus and act a structural attachment site for the DNA loops during interphase.

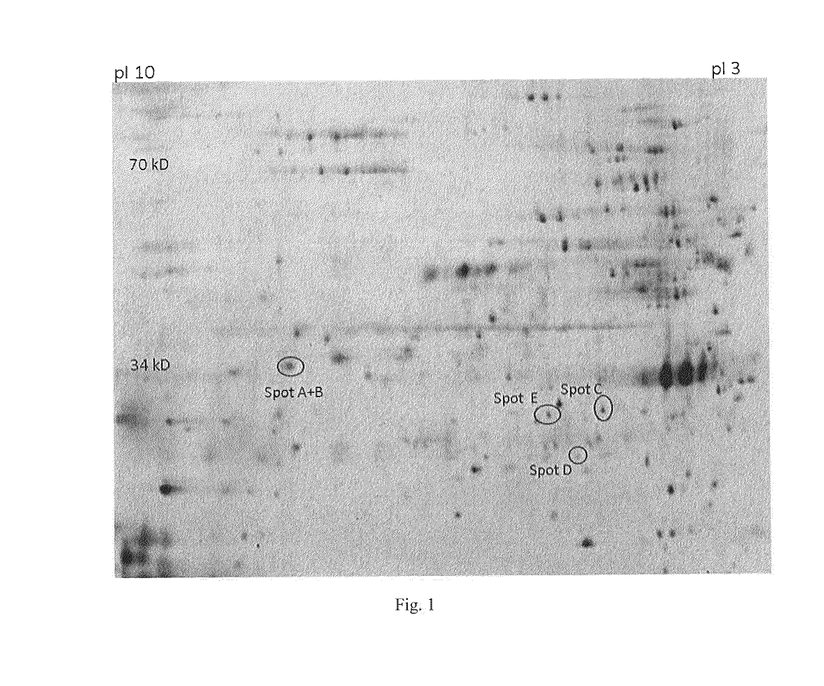

In various embodiments, the one or more additional markers are selected from the group consisting of hsp27, estrogen receptor, progesterone receptor, EGFR, Her2, circulating DNA, circulating RNA, circulating tumor cells, upa/PAI1, miRNA, ki67, Bone Sialoprotein, CA15-3, CA27.29, CEA, P53, Cathepsin D, Cyclin E, Vitronectin, Vimentin, S100, MMP11, CTSL2, STK15, Survivin, Cyclin B1, MYBL2, GSTM1, BAG1, ITIH4, C3a-complement, GCDFB-15, ApoD, alpha-1-acid glycoprotein, the protein in Spot C of the 2D-gel-electrophoresis (FIG. 1, 25 kDa, pI 4), the protein in Spot E of the 2D-gel-electrophoresis (FIG. 1, 20 kDa, pI 4,5), CA15-3, CA27.29, CEA, the protein in spot F of the 2D-gel-electrophoresis (FIG. 1, 20 kDa, pI 4), CEACAM1, tissue inhibitors of metalloproteinases (TIMP)-3, Calgranulin A, LyGDI, RhoA, Profilin, and Apo-C1.

In one embodiment, the one or more additional markers are selected from the group consisting of in a hsp27, estrogen receptor, the Protein in Spot C of the 2D-gel-electrophoresis (FIG. 1, 25 kDa, pI 4), and the Protein in Spot E of the 2D-gel-electrophoresis (FIG. 1, 20 kDa, pI 4,5).

If one or more additional marker levels are determined, this may increase the accuracy of the method. However, the predictive value of the markers may not be only related to an increase in level, but also to a decrease. This means that the decrease of certain marker levels may have diagnostic value in the methods of the present invention. “Decrease”, in this context, means that the level of a given marker is reduced compared to its normal level, for example its level in a healthy individual or a patient not afflicted by breast carcinoma. This includes that a protein normally present and detectable, is absent or not detectable any more.

Exemplary markers, the level of which is decreased in breast carcinoma include, but are not limited to protein F (FIG. 1, 20 kDa, pI 4), CEACAM1 and Tissue inhibitors of metalloproteinases (TIMP)-3, Calgranulin A, LyGDI, RhoA, Profilin, and Apo-C1.

The other markers listed above, are usually increased in breast carcinoma.

As noted above, including the determination of additional marker levels in the methods of the invention may increase the accuracy of the method. For example, in a method of diagnosing breast carcinoma in a patient, if the level of Calponin-h2 and/or CALML5 is increased and the level of the at least one or more additional markers is increased or decreased, said patient is diagnosed with breast carcinoma with a higher accuracy. This is also applicable to the methods of determining a prognosis or the efficacy of a treatment regimen.

In various embodiments of the invention, the sample is a biological sample, for example a body fluid, cell or tissue sample. Body fluids comprise, but are not limited to blood, blood plasma, blood serum, breast milk, cerebrospinal fluid, cerumen (earwax), endolymph and perilymph, gastric juice, mucus (including nasal drainage and phlegm), peritoneal fluid, pleural fluid, saliva, sebum (skin oil), semen, sweat, tears, vaginal secretion, nipple aspirate fluid, vomit and urine. In certain embodiments of the methods detailed above, the body fluid is selected from the group consisting of blood, serum, plasma, urine, and saliva. The tissue sample may be breast tissue and the cell sample may comprise cells from breast tissue.

In certain embodiments, the sample may be subjected to processing before the marker levels are determined. In one embodiment, the sample can, for example, be fractionated to enrich the nuclear matrix proteins (NMP).

NMPs may be enriched from any biological sample. In certain embodiments NMPs are enriched from cells, tissue or body fluid. The term “enriched” means that at least some NMP are present in higher concentrations in the enriched sample compared to the non-enriched sample. NMP preparations may be prepared by well known methods in the art such as detergent and urea extraction (Getzenberg et al., Cancer Res, 51: 6514-6520, 1991). An NMP preparation that is enriched in NMPs may additionally contain other proteins, i.e. proteins that are not part of the nuclear matrix.

In some embodiments the patient is a mammal, preferably a human.

Generally, the term “mammal”, as used herein, comprises human, monkeys, pigs, cows, cats, dogs, guinea pigs, rabbits, mice, sheep, goats and horses.

For the detection of the markers of the present invention specific binding partners may be employed. In some embodiments, the specific binding partners are useful to detect the presence of a marker in a sample, wherein the marker is a protein or RNA. The marker and its binding partner represent a binding pair of molecules, which interact with each other through any of a variety of molecular forces including, for example, ionic, covalent, hydrophobic, van der Waals, and hydrogen bonding. Preferably, this binding is specific. “Specific binding” means that the members of a binding pair bind preferentially to each other, i.e. usually with a significant higher affinity than to non-specific binding partners. The binding affinity for specific binding partners is thus usually at least 10-fold, preferably at least 100-fold higher than that for non-specific binding partners.

Exemplary binding partners for the markers of the invention are selected from the group consisting of antibodies, antibody fragments and variants, molecules with antibody-like properties, such as lipocalin muteins or Spiegelmers or aptamers. Antibody fragments and variants include Fv fragments, linear single chain antibodies and the like all of which are known to those skilled in the art.

In various embodiments of the methods detailed above, determining the level of Calponin-h2 and/or CALML5 comprises determining the amount of protein present in a sample. In other embodiments, determining the level of a marker includes determining the expression level of Calponin-h2 and/or CALML5, for example on the RNA level. Generally, determining the level of a marker comprises determining the mRNA level and/or protein level of a maker.

Accordingly, in some embodiments the level of at least one or more markers is determined on mRNA level. In further embodiments the level of at least one or more markers is determined on protein level. In various embodiments, at least one or more markers are determined at mRNA level and at least one or more markers are determined at protein level.

If a marker is determined on mRNA level, the mRNA may be the mRNA transcript, a 5′- and/or 3′-truncated mRNA or spliced mRNA forms.

If a marker is determined on protein level, the protein may be the full length protein or a fragment thereof. The protein fragment may be a truncated protein, i.e. lack one or more amino acids at the N-terminus or C-terminus or both. This may be due to post-translational processing or due to the action of proteases present in the cell or the sample. The markers determined in the methods of the invention thus also include naturally occurring fragments, preferably immunogenic fragments. Also, the protein may be posttranslationally modified, e.g., phosphorylated, hydroxylated, glycosylated, N-glycosylated, O-glycosylated, ubiquitinylated, acetyated, methylated, prenylated or sulphated.

In certain embodiments of the methods of the invention, the levels of at least 2, 3, 4, 5, 6, 7, 8, 9, 10, 20, 50 or 100 or more markers are determined.

In various embodiments of the methods of the present invention, determining the level of Calponin-h2 and/or CALML5 comprises determining the Calponin-h2 and/or CALML5 protein level in a sample.

It is understood that in certain embodiments the methods comprise determining the level of Calponin-h2 and CALML5 which comprises determining the Calponin-h2 and CALML5 protein level. In alternative variants of these embodiments, the level of one or more further markers is determined. In some of these embodiments, the level of the one or more markers is determined at protein and/or mRNA level. Accordingly, in some embodiments the methods involve the determination of the level of the markers on protein level only.

The methods detailed above, wherein the level of at least one or more markers is determined on protein level comprise in some embodiments the determination of the protein level by an immunoassay, mass spectrometry, chromatography, Western Blot, or gel electrophoresis.

In some embodiments, the immunoassay may be, but are not limited to an Enzyme-linked Immunosorbent Assay (ELISA), Western blot, agglutination test, biotin/avidin type assays, radioimmunoassays, immunoelectrophoresis and immunoprecipitation. The reactions generally include revealing labels such as fluorescent, chemiluminescent, radioactive, enzymatic labels or dye molecules, or other methods for detecting the formation of a complex between the antigen and the antibody or antibodies reacted therewith These and further immunoassays are well known in the art (David Wild (Ed.): The Immunoassay Handbook. 3^{rd} ed. Elsevier Science Publishing Company, Amsterdam 2005).

The aforementioned assays may involve separation of unbound protein in a liquid phase from a solid phase support to which antigen-antibody complexes are bound. Solid supports which can be used in the practice of the invention include substrates such as nitrocellulose (e.g., in membrane or microtiter well form); polyvinylchloride (e.g., sheets or microtiter wells); polystyrene latex (e.g., beads or microtiter plates); polyvinylidine fluoride; diazotized paper; nylon membranes; activated beads, magnetically responsive beads, and the like.

More particularly, an ELISA method can be used, wherein the wells of a microtiter plate are coated with an antibody against the protein to be tested. A biological sample containing or suspected of containing the marker is then added to the coated wells. After a period of incubation sufficient to allow the formation of antibody-antigen complexes, the plate(s) can be washed to remove unbound moieties and a detectably labeled secondary binding molecule added. The secondary binding molecule is allowed to react with any captured sample marker protein, the plate washed and the presence of the secondary binding molecule detected using methods well known in the art.

In certain embodiments of the above detailed methods, if the determination is via mass spectrometry, the mass spectrometry may be selected from the group comprising MS measurements using EI, CI, ESI, APLI, APPI and APCI.

The marker determination on protein level employing chromatography may be selected from the group comprising liquid chromatography, HPLC, FPLC, Smart chromatography, gel chromatography, size exclusion chromatography, reverse phase chromatography and ion-exchange chromatography (Introduction to Modern Liquid Chromatography, Lloyd R. Snyder, Wiley, 2009).

In various embodiments, if the marker is detected via gel electrophoresis, the gel electrophoresis may be selected from the group, but not limited to agarose gel electrophoresis, sodium dodecyl sulfate poly acrylamide gel electrophoresis (SDS-PAGE), 2D-gel electrophoresis, native gel electrophoresis and quantitative preparative native continuous polyacrylamide gel electrophoresis (QPNC-PAGE).

Of course, in certain embodiments of the methods of the present invention at least two determination methods may be coupled to each other in a subsequent manner. In a variant, a gel electrophoresis may be followed by a mass spectroscopic analysis. Alternatively, a gel electrophoresis may be followed by a Western Blot, a chromatography may be followed by a mass spectroscopic analysis, a chromatography may be followed by an immune assay, e.g. an ELISA.

In further embodiments of the methods of the present invention, determining the level of Calponin-h2 and/or CALML5 comprises determining the Calponin-h2 and/or CALML5 mRNA level in a sample. In alternative variants of these embodiments, the level of one or more further markers is determined. In some of these embodiments, the level of the one or more markers is determined at protein and/or mRNA level. Accordingly, in some embodiments the methods involve the determination of the level of the markers on RNA level only.

Where in the methods detailed above a marker is determined on mRNA level, the RNA level may be determined by PCR, gel electrophoresis and/or Northern Blot.

In case the marker level is determined on the RNA level, the detection reagent may be a nucleic acid molecule, such as an oligonucleotide. The oligonucleotide may be a nucleic acid probe that may be labeled to allow detection or may be an oligonucleotide primer that allows amplification of the target molecule.

In a further aspect, the present invention relates to a kit for use in a method as detailed above, wherein the kit comprises reagents for determining the level of Calponin-h2 and/or CALML5 in a sample. In certain embodiments, the reagents for determining the level of Calponin-h2 and/or CALML5 in a sample are antibodies and/or oligonucleotides. In some of the above embodiments, the kit comprises reagents for determining the level of Calponin-h2 and CALML5 in a sample. Also disclosed herein are kits comprising reagents for determining the level of Calponin-h2 and CALML5 and at least one or more further markers in a sample. In one embodiment, the kit comprises further reagent for the detection of at least 1, 2, 3, 4, 5, 6, 7, 8, 9, 10, 20, 30, 40, 50 or 100 further markers. Specifically, the kit may further comprise reagents for the determination of markers selected from the group comprising hsp27, estrogen receptor, the protein in spot C of the 2D-gel-electrophoresis (FIG. 1, 25 kDa, pI 4), the protein in spot E of the 2D-gel-electrophoresis (FIG. 1, 20 kDa, pI 4,5), protein F (FIG. 1, 20 kDa, pI 4), CEACAM1, tissue inhibitors of metalloproteinases (TIMP)-3, Calgranulin A, LyGDI, RhoA, Profilin, Apo-C1.

In addition, the present invention provides a marker panel comprising Calponin-h2 and CALML5 for use in a method detailed above. In certain embodiments, the panel comprises at least one or more further markers. In one embodiment, the marker panel comprises at least 1, 2, 3, 4, 5, 6, 7, 8, 9, 10, 20, 30, 40, 50 or 100 further markers. Specifically, the panel may further comprise markers selected from the group comprising hsp27, estrogen receptor, the Protein in Spot C of the 2D-gel-electrophoresis (FIG. 1, 25 kDa, pI 4), the Protein in Spot E of the 2D-gel-electrophoresis (FIG. 1, 20 kDa, pI 4,5), protein F (FIG. 1, 20 kDa, pI 4), CEACAM1 and Tissue inhibitors of metalloproteinases (TIMP)-3, Calgranulin A, LyGDI, RhoA, Profilin, Apo-C1.

### EXAMPLES

### Example 1

### Tissue Samples and Cell Lines

Breast tissues were obtained from the Department of Obstetrics and Gynecology at the University of Bonn Medical Center as well as the Department of Plastic and Aesthetic Surgery at the University of Bonn Medical Center in cooperation with the tissue bank of the Center for Integrated Oncology Cologne-Bonn. For all specimens, a histopathological workup was performed by an experienced, board-certified breast pathologist. The characteristics of patients and tumors for two-dimensional gel-electrophoresis as well as verification experiments by one-dimensional Immunoblot are summarized in Table 1 and 2. None patient received neo-adjuvant treatment prior to surgery. The investigations conformed to the principles outlined in the Declaration of Helsinki and were performed with permission by the responsible Ethics Committee of the School of Medicine, University of Bonn.

TABLE 1
Patient and tumor characteristics.
Cancer patient tissues used in 2-D gels and immunoblot analyses

| | Age at | | | | | | |
|---|---|---|---|---|---|---|---|
| Sample | diagnosis | Menopause | Histology | T-stage | Nodal-status | Metastasis | Grade |
| Tumor 1 | 50 | postmeno | invasive-ductal | 1 | 0 | 0 | 2 |
| Tumor 2 | 72 | postmeno | invasive-ductal | 1 | 0 | 0 | 2 |
| Tumor 3 | 74 | postmeno | invasive-ductal | 4 | 1 | 0 | 3 |
| Tumor 4 | 63 | postmeno | invasive-ductal | 1 | 0 | 0 | 2 |
| Tumor 5 | 58 | postmeno | invasive-ductal | 2 | 1 | 0 | 3 |
| Tumor 6 | 65 | postmeno | invasive-ductal | 1 | 1 | 0 | 2 |
| Tumor 7 | 66 | postmeno | invasive-ductal | 1 | 3 | 0 | 2 |
| Tumor 8 | 64 | postmeno | invasive-ductal | 3 | 3 | 0 | 3 |
| Tumor 9 | 41 | premeno | invasive-ductal | 2 | 0 | 0 | 2 |
| Tumor 10 | 48 | premeno | invasive-ductal | 2 | 1 | 0 | 3 |
| Tumor 11 | 59 | postmeno | invasive-ductal | 1 | 0 | 0 | 3 |
| Tumor 12 | 41 | premeno | invasive-ductal | 1 | 0 | 0 | 2 |
| Tumor 13 | 56 | postmeno | invasive-ductal | 1 | 1 | 0 | 3 |
| Tumor 14 | 70 | postmeno | invasive-ductal | 1 | 2 | 0 | 3 |
| Tumor 15* | 59 | postmeno | invasive-ductal | 1 | 1 | 0 | 2 |
| Tumor 16* | 63 | postmeno | invasive-lobular | 1 | 1 | 0 | 2 |
| Tumor 17* | 50 | premeno | invasive-mucinous | 2 | 3 | 0 | 3 |

*sample was used for protein validation by one-dimensional immunoblot

TABLE 2
Characteristics of human healthy and benign controls.
Benign and healthy control tissues used in 2-D gels and
immunoblot analyses

| | Age at | | |
|---|---|---|---|
| Sample | diagnosis | Menopause | Histology |
| Control 1 | 48 | postmeno | Healthy breast tissue |
| | | | (Breast Reduction) |
| Control 2 | 20 | premeno | Healthy breast tissue |
| | | | (Breast Reduction) |
| Control 3 | 19 | premeno | Fibroadenoma |
| Control 4 | 18 | premeno | Fibroadenoma |
| Control 5* | 48 | postmeno | Healthy breast tissue |
| | | | (Breast Reduction) |
| Control 6* | 53 | postmeno | Healthy breast tissue |
| | | | (Breast Reduction) |

*sample was used for protein validation by one-dimensional immunoblot

To verify the epithelial character of specific protein spots the NMP composition of different breast cancer cell lines was investigated. Breast cancer cell lines (HCC1937, BT474, SkBr3 and MCF7) as well as MCF10a, representing normal human epithelial breast cells, were obtained from the American Type Culture Collection and cultured under appropriate conditions.

Blood samples of the following set of patients were subjected to ELISA-based Calponin-h2 detection:

TABLE 3
Characteristics of early breast cancer and control patients of whom serum
samples were taken for an ELISA-based Calponin-2 determination. DCIS =
ductal carcinoma in-situ, T = T-stage, N = Nodal-status, M = Metastasis,
G = Grade, ER = estrogen receptor, PR = progesterone receptor, and
HER2 = human epidermal growth factor receptor 2.

| Breast | | | | | | | | | |
|---|---|---|---|---|---|---|---|---|---|
| cancer | age | Histology | T | N | M | G | ER | PR | Her2 |
| P 1 | 53 | DCIS | Tis | 0 | 0 | 2 | pos | pos | neg |
| P 2 | 64 | DCIS | Tis | 0 | 0 | 3 | pos | pos | neg |
| P 3 | 46 | invasive ductal | 1c | 1mi | 0 | 3 | pos | pos | neg |
| P 4 | 80 | invasive ductal | 2 | 2a | 0 | 3 | neg | neg | neg |
| P 5 | 49 | ductulolobular- | 1b | 2a | 0 | 2 | pos | pos | neg |
| | | invasive | | | | | | | |
| P 6 | 48 | invasive ductal | 2 | 0 | 0 | 3 | pos | pos | pos |
| P 7 | 58 | invasive ductal | 1c | 1a | 0 | 3 | pos | pos | pos |
| P 8 | 84 | invasive ductal | 2 | 0 | 0 | 2 | pos | pos | neg |
| P 9 | 30 | invasive ductal | 1c | 0 | 0 | 2 | pos | pos | neg |
| P 10 | 74 | ductal multicentric/ | 1c | 0 | 0 | 2 | pos | pos | neg |
| | | invasive tubular | | | | | | | |
| P 11 | 37 | invasive ductal | 2 | 0 | 0 | 3 | pos | pos | pos |

| Control | |
|---|---|
| women | age |
| K 1 | 30 |
| K 2 | 39 |
| K 3 | 44 |
| K 4 | 61 |
| K 5 | 53 |

### Example 2

### Methods

### Nuclear Matrix Preparation

Nuclear matrix proteins were extracted according to techniques as previously described (Getzenberg et al., Cancer Res, 51: 6514-6520, 1991). Briefly, the frozen breast cancer samples were pulverized in a Micro-Dismembrator (B Braun Biotech International) and transferred in a buffer containing 0.5% Triton X-100 (Carl Roth, Germany) and 2 mM Ribonucleoside vanadyl complexes (Sigma-Aldrich, USA) to release lipids and soluble proteins. Afterwards, the solution was filtered through a 350 μm nylon mesh and underwent treatment with DNase as well as RNase-A to remove the soluble chromatin and RNA. The remaining fraction, comprising intermediate filaments and NMPs, was disassembled with 8M urea and the insoluble components (mainly carbohydrates and extracellular matrix) were pelleted. After dialyzing the urea out, the intermediate filaments were allowed to reassemble and were subsequently removed by centrifugation. In a final step, the NMPs were precipitated in ethanol and resolved in 2-D sample buffer (9M Urea, 4% Chaps, 82 μM TBP, 0.4% Ampholyte) or phosphate buffered saline (PBS). The described reactions, besides the digestion with DNase and RNase, were performed on ice. All solutions contained 1 mM PMSF to inhibit serine proteases. To remove potentially interfering contaminants a sample cleanup was performed (ReadyPrep 2-D Cleanup; Bio-Rad, USA). The protein concentration was quantified by a Reducing Agent Compatible Microplate BCA Protein Assay Kit (Thermo Scientific, USA) with bovine serum albumin as a standard. The final pellet containing NMPs represents <1% of the total cellular proteins.

### High-Resolution Two-Dimensional Gel Electrophoresis

Isoelectric focusing was carried out in a PROTEAN IEF Cell (Bio-Rad, USA) according to the manufacturer's instructions. Samples containing 100 μg of NMP were added to 24 cm immobilized ph gradient (IPG) strips (Bio-Rad, USA) in the range of IP 3-10. After 16 h of passive rehydration two wet paper wicks were inserted between the IPG strip and the electrode. A gradient at 200V for 2 h, 500V for 2 h, 2000V for 3.5 h and 8000V for 2 h was applied to the IPG strips. The temperature throughout this process was maintained at 20° C. After isoelectric focusing the IPG strips were equilibrated in EQ-buffer I (6 M Urea, 0.375M Tris/HCl pH 8.8, 20% Glycerol, 2% SDS, 2% DTT) and EQ-buffer II (6 M Urea, 0.375 MTris/HCl pH 8.8, 20% Glycerol, 2% SDS, 2.5% Iodoacetamide) for 15 minutes each. The equilibrated strips were loaded onto 10% Polyacrylamide gels and overlaid with 1% agarose. The separation in the second dimension was performed in a Investigator 2-D Electrophoresis System (Genomic Solutions, USA) until the bromophenol blue front reached the end of the gel. After SDS-PAGE the separated NMPs were visualized by silver staining (SILVERQUEST Silver Staining Kit; Invitrogen, USA). The gels were analyzed using the PDQuest 2D Analyzing Software (Bio-Rad, USA). Only clear and reproducibly identical spots in all of the gels were included in the analysis.

### Mass Spectrometric Analysis

Spots of interest were automatically cut from silver stained gels and processed using a Trypsin Profile IGD Kit (Sigma-Aldrich, USA) following the manufacturer's instructions. For analysis eluted peptides were separated using an Ultimate 3000 LC system (Dionex-LC Packings, Germany). Samples were loaded onto a monolithic trapping column (PepSwift, 200 μm*5 mm) by the loading pump of the system operating at 10 μL/min, and 0.1% Heptafluorobutyric acid in water was used as mobile phase. After 5 min, valve was switched and the sample was eluted onto the analytical separation column (PepSwift monolithic capillary column, 200 μm 50 mm), using a flow rate of 500 mL/min. The mobile phases used were H20/0.1% Formic acid (v/v) for buffer A and 100% ACN/0.1% Formic acid (v/v) for buffer B. Peptides were resolved by gradient elution using a gradient of 5-50% buffer B over 20 min, followed by a gradient of 50-90% buffer B over 1 min. After 5 min at 90% B the gradient returned to 5% buffer B preparing for the next run. Column effluent was monitored using a 3 nL UV flow cell (214 nm).

Mass spectrometric analysis was done via online ESI-MS/MS using an HCTUItra ion trap mass spectrometer (Bruker Daltonics, Germany). All measurements were carried out in positive ion mode. MS-spectra were acquired in standard-enhanced mode between 300 to 2000 m/z at a rate of 8,100 m/z/sec. Fragmentation of peptides from MS-spectra using CID was done in Auto-MS2 mode, selecting precursor ions according to the following parameters: number of precursor ions=5, minimal ion intensity=10,000, ion excluded after 2 spectra, exclusion release after 1 min. MS2 data acquisition was done in ultrascan mode with a scan range of 50-3000 m/z at a scan speed of 26,000 m/z/sec.

Raw MS data for each LC run were processed using DataAnalysis™ version 4.0. The spectrum was screened for compounds using the software's AutoMS/MS search feature applying following parameters: intensity threshold=10,000; max number of compounds=500; retention time=0.4. Identified compounds were subsequently deconvoluted and exported for protein database comparison with BioTools™ version 3.1. In BioTools™ the exported compounds were run against an in-house SwissProt v51.6 database using the Mascot 2.2.02 algorithm. The searches were carried out using the following parameters: enzyme=trypsin; missed cleavages=1; taxonomy 32 All entries; variable modifications=oxidation (M) and carbamidomethylation (C); peptide tolerance=300 ppm; MS/MS tolerance=1.1 Da; significance threshold p=0.05.

### Nuclear and Cytoplasmic Extraction

To exclude a cytoplasmic expression of Calponin-h2 by one-dimensional Immunoblot, a NE-PER Nuclear and Cytoplasmic Extraction Kit (Thermo Scientific, USA) was used for the preparation of nuclear and cytoplasmic extracts. The protein concentration was quantitated by a Reducing Agent Compatible Microplate BCA Protein Assay Kit (Thermo Scientific, USA) with bovine serum albumin as a standard.

### One-Dimensional Immunoblot

To validate the Calponin-h2, which has been identified by MS analysis NMP-extracts of human breast cancer and healthy breast tissue and cell lines were studied. One-Dimensional Immunoblot was performed on NMP-extracts of breast cancer patients and healthy controls, which had not been used for protein separation by two-dimensional gel electrophoresis. To outline the specificity of the identified proteins we also investigated different histological breast cancer types (ductal, lobular and mucinous). Ten micrograms of NMP per sample were separated by 12% SDS-PAGE (Invitrogen, USA), transferred onto nitrocellulose membranes and blocked overnight in BSA in TBS with 0.5% Tween-20. The following day membranes were incubated with anti-Calponin-h2 1:200 (No: sc-16608; Santa-Cruz, USA) for 2 h at room temperature. Anti-Lamin A/C 1:200 (No: sc-56140; Santa-Cruz, USA) was used as a nuclear and anti-α-Tubulin 1:200 (No: sc-58666; Santa-Cruz, USA) as a cytoplasmic control. Species-appropriate fluorescently conjugated secondary antibodies were applied for 1 h at room temperature. Membranes were analyzed using an Odyssey Infrared Imaging System (LI-COR, Australia).

### Enzyme-Linked Immunosorbent Assay (ELISA) for CNN2

The human sera of the patients listed in Table 3 above were diluted 1:8 in LowCross Buffer Classic (Candor, Germany) and analyzed using an CNN2 assay kits (USCN Life Science Inc., China) according to the manufacturer's instructions. Briefly, the microtiter plates provided with the kit were pre-coated with a monoclonal antibody against CNN2. Reference standards and samples were added to the appropriate wells and incubated for 2 hours prior to incubation with a CNN2 specific biotin-conjugated polyclonal antibody (1 hour). Next, Avidin-Horseradish-Peroxidase (HRP) was added to each well and incubated for 30 minutes. Afterwards, each well was supplemented with TMB substrate. After 20 minutes the enzyme-substrate reaction was stopped by the addition of a sulphuric acid solution and the absorbance was recorded at 450 nm±10 nm using a Safire microplate reader (Tecan, Switzerland). All incubation steps were performed at 37° C.

### Statistical Methods

All receiver operating characteristic curves, areas under the curve, and 95% confidence intervals were calculated with Graph Pad Prism Version 5. In order to analyze differences between the patient groups, a one way analysis of Variance (ANOVA) was performed with the Dunnett's post hoc test. Statistical significance was assumed at p<0.05.

### Example 3

### Identification of Markers of Breast Cancer

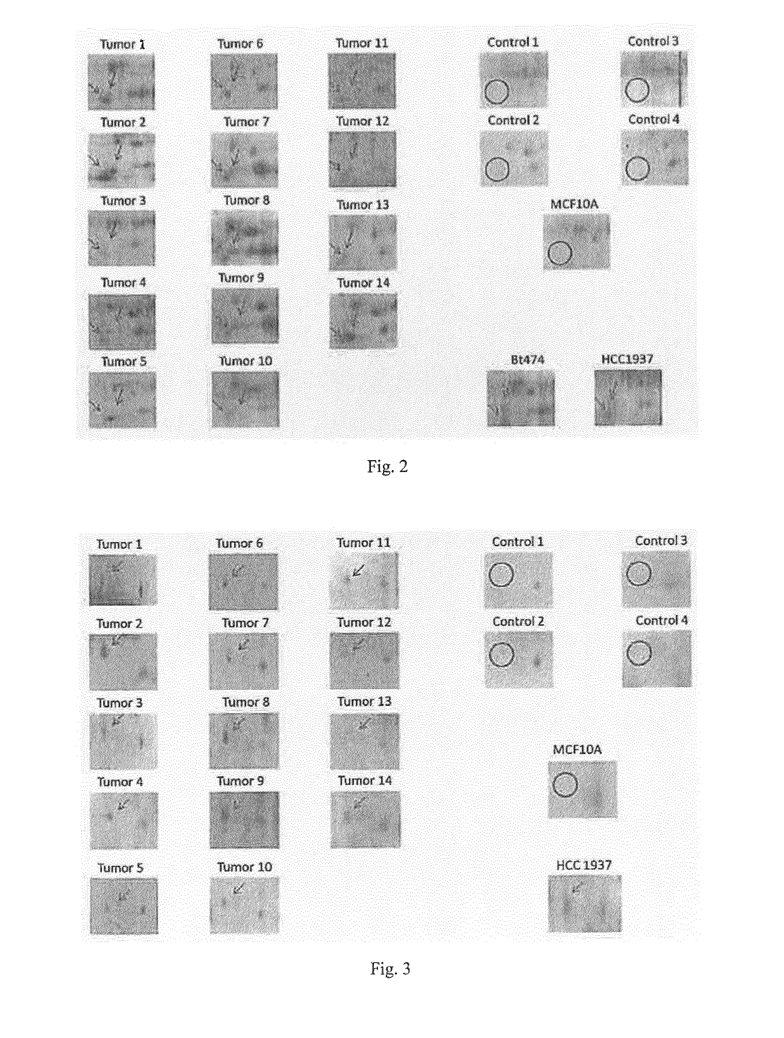

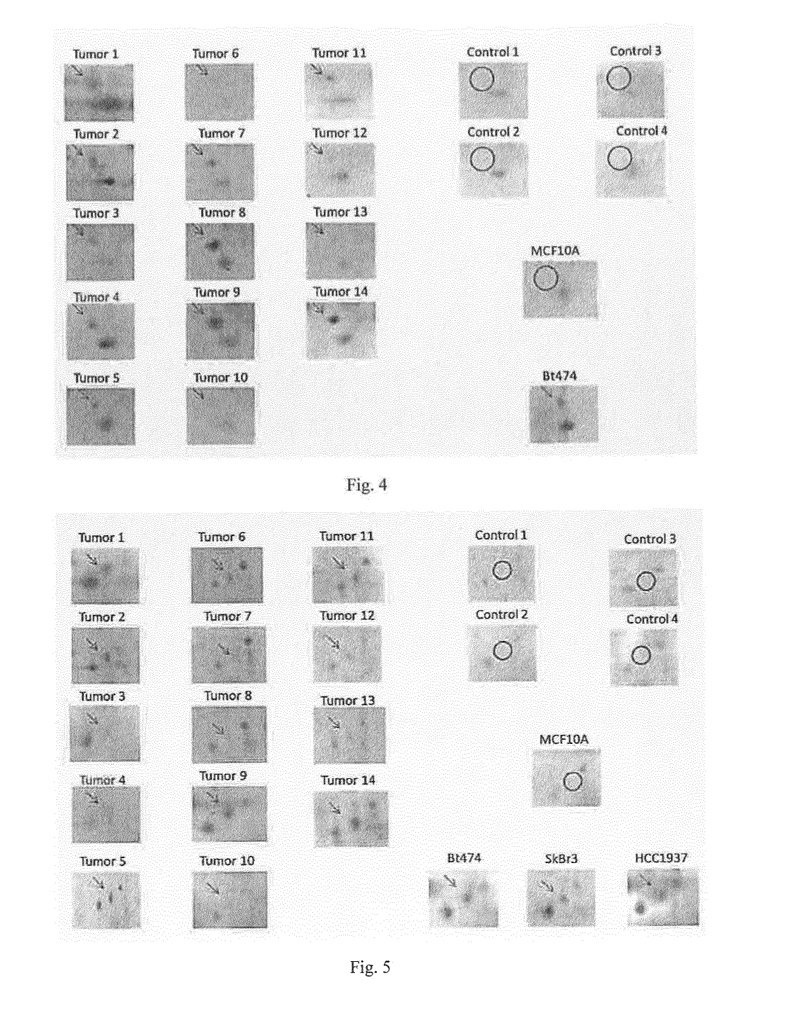

Human breast cancer tissues of 14 different patients with ductal-invasive breast cancer as well as four non-malignant controls (2 fibroadenoma and 2 healthy controls) were investigated. Therefore, the NMP were extracted and separated by high-resolution two-dimensional gel electrophoresis. FIG. 1 shows a representative two-dimensional gel (2-D gel) of nuclear matrix proteins in human breast cancer. Performing a computer-based comparison of all 2-D gels by PDQuest 2D Analyzing Software five protein spots (A,B,C,D,E) have been identified to be present in all human cancer tissues but not in any control (Table 4). These spots were also present in the investigated breast cancer cell lines, demonstrating the epithelial source of the found proteins spots (FIGS. 2-5). In addition, one protein spot (F) was exclusively found in healthy breast tissue but absent in fibroadenoma and human breast cancer. To clarify the identity of each specific protein by MS analysis, up to four protein spots were pooled prior to trypsin digestion to enhance signal intensity and therefore protein identification. As shown in Table 5 protein spots A, B, D were successfully identified. The breast cancer specific protein spots came out to be Calponin-h2 (CNN-2; Spot A), Calmodulin-like protein 5 (CALML5; Spot B) and heat shock protein (hsp) beta 1 (hsp27; Spot D). For spots C, E and F no protein identification was possible by MS analysis.

TABLE 4
NMP present in human breast cancer tissue and human controls
(Values are given as percentage). Investigating the expression of
nuclear matrix proteins in human breast cancer (n = 14),
fibroadenoma (n = 2) and healthy controls (n = 2) by
high-resolution two-dimensional gel electrophoresis and silver
staining, protein spots A-E appeared to be specific for human
breast cancer, whereas protein spot F was only present in the
healthy controls

| protein spot | breast cancer | fibroadenoma | healthy control |
|---|---|---|---|
| A | 100% (14/14) | 0% (0/2) | 0% (2/2) |
| B | 100% (14/14) | 0% (0/2) | 0% (2/2) |
| C | 100% (14/14) | 0% (0/2) | 0% (2/2) |
| D | 100% (14/14) | 0% (0/2) | 0% (2/2) |
| E | 100% (14/14) | 0% (0/2) | 0% (2/2) |
| F | 0% (0/14) | 0% (0/2) | 100% (2/2) |

TABLE 5
Protein identification by Mass analysis.
Specific nuclear matrix proteins in human breast cancer

| protein | | MW | | MOWSE | Sequence |
|---|---|---|---|---|---|
| spot | protein | (Da) | pI | score | coverage (%) |
| A | Calponin-h2 | 33675 | 6.95 | 140 | 12 |
| B | Calmodulin-like | 15883 | 4.34 | 588 | 76 |
| | protein 5 | | | | |
| C | unidentified | 25000* | 4* | — | — |
| D | hsp beta1 | 22768 | 5.98 | 184 | 20 |
| E | unidentified | 20000* | 4.5* | — | — |

Nuclear matrix protein only found in healthy breast tissue

| protein | | MW | | MOWSE | Sequence |
|---|---|---|---|---|---|
| spot | identified protein | (kDa) | pI | score | coverage (%) |
| F | unidentified | 20000* | 4* | — | — |

*approximate MW and pI in 2D-gel.

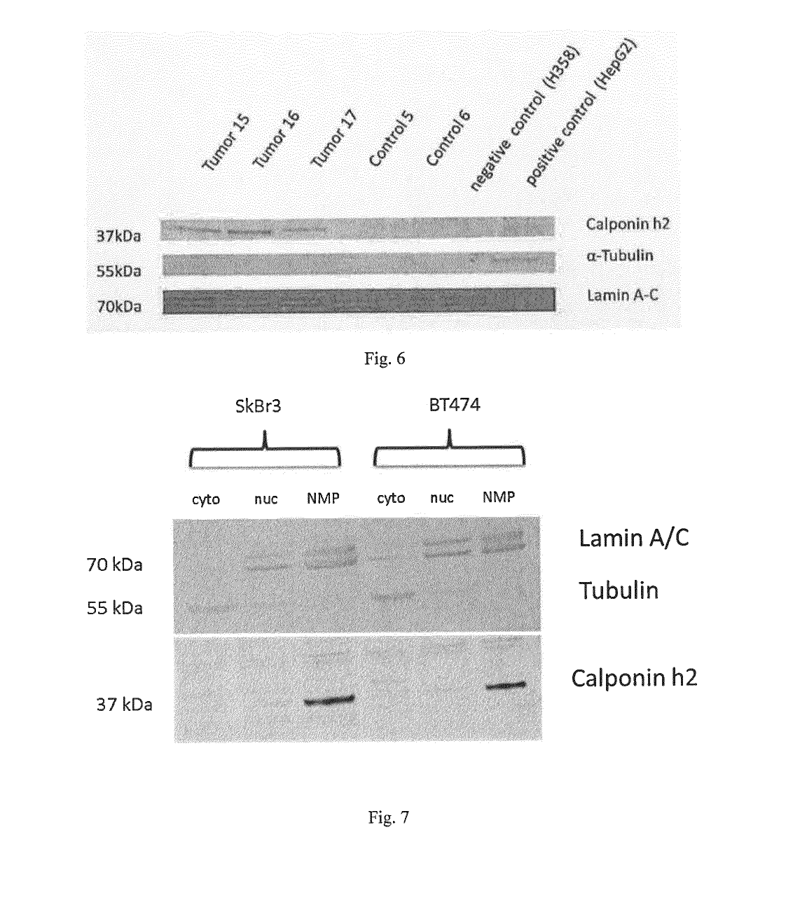

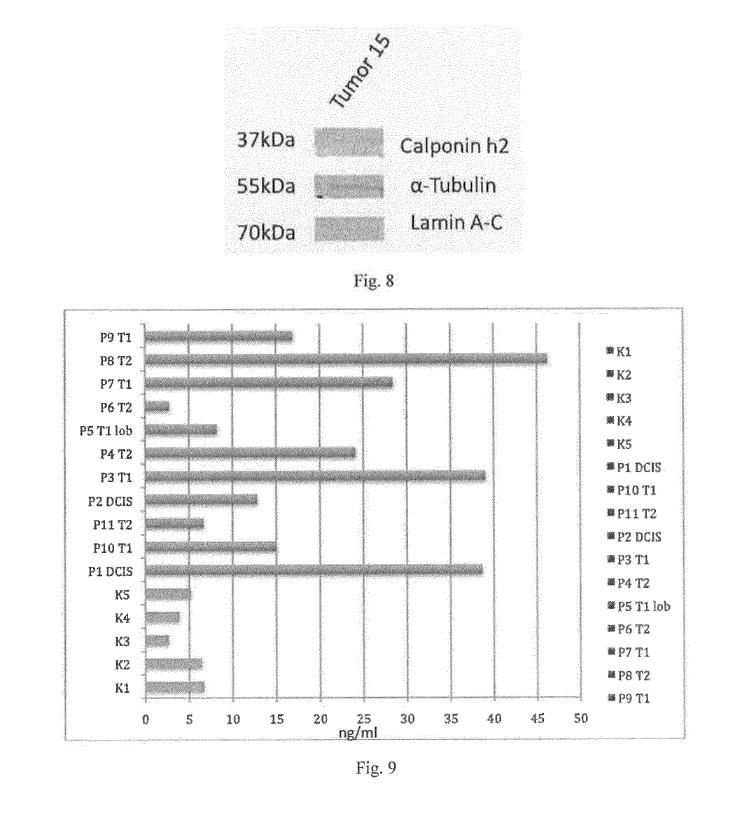

One-dimensional Immunoblot with commercially available antibodies against CNN-2 was performed to validate the results of MS analysis. As shown in FIG. 6 the antibody against CNN-2 detected a protein band at 37 kDa in the NMP-fraction of histological different human breast cancer entities (ductal, lobular and mucinous) but not in healthy human breast tissue. This band was also present in the NMP-fraction of the investigated breast cancer cell lines (FIG. 7). To rule out the possibility of a contamination of the nuclear matrix protein extraction with cytoplasmic proteins we performed one-dimensional immunoblot analysis in the cytoplasmic and nuclear matrix protein fractions. A polyclonal α-tubulin antibody specific for cytoplasmic α-tubulin (approximately 51 kD) was used as a control and identified α-tubulin exclusively in the cytoplasmic protein fraction. The nuclear matrix protein fractions did not contain a band recognized by the α-tubulin antibody. These results suggest that the contamination of these fractions with cytoplasmic proteins is unlikely. These results demonstrate, that a cytoplasmic expression of CNN-2 is not seen (FIG. 8).

The upregulation of Calponin-h2 in human breast cancer and also its nuclear localization has not been described before. The detection of calponin h2 in breast cancer cell lines confirms the epithelial expression and excludes potential impurites by, e.g., fibroblasts or endothelial cells.

### Example 4

### Confirmation of Calponin-h2 as a Breast Cancer Marker

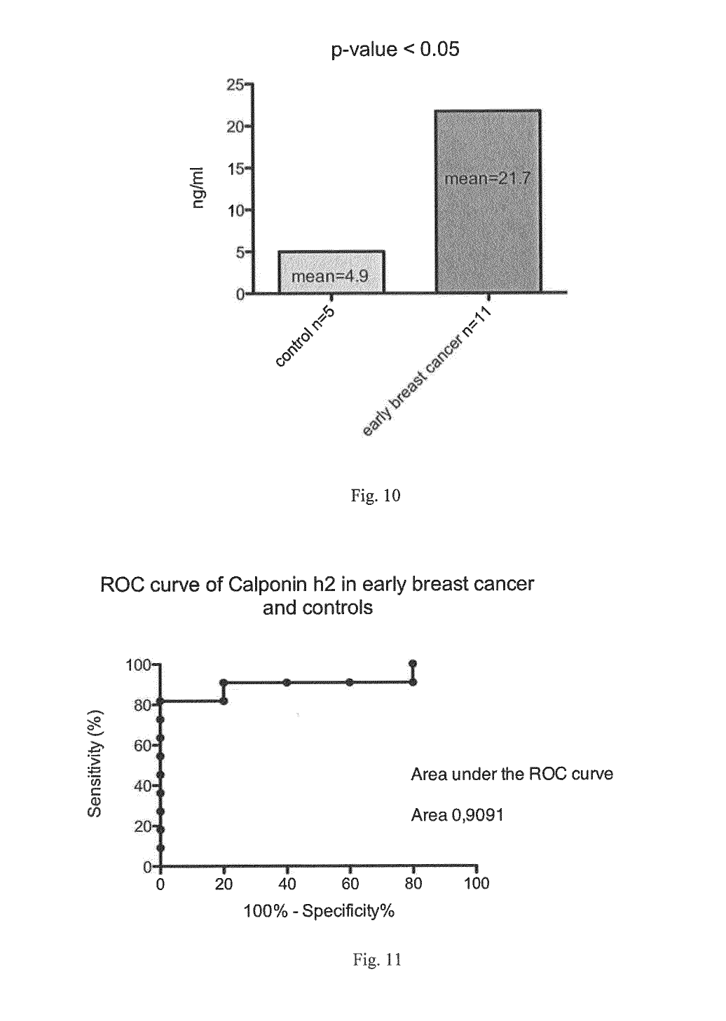

In order to assess the suitability of Calponin-h2 as a marker for the prediction/detection of breast cancer, serum samples of breast cancer patients and healthy controls (cf. Table 3) were analyzed using the Calponin-h2 ELISA protocol, as described above. FIG. 9 shows the amount of Calponin-h2 detected in each serum sample. Thus, ELISA is suitable for Calponin-h2 detection in serum samples. Furthermore, a statistical analysis of the data revealed that the Calponin-h2 serum levels are significantly elevated in breast cancer patients compared with samples obtained from healthy controls (cf. FIG. 10). The statistical significance of this difference and the reliability of the ELISA to detect the difference was confirmed using the ROC curve (FIG. 11).

TABLE 6
Results of the ROC curve statistical analysis of FIG. 11.

| Area under the ROC curve | |
|---|---|
| Area | 0.9091 |
| Std. Error | 0.07745 |
| 95% confidence interval | 0.7573 to 1.061 |
| P value | 0.01083 |
| Data | |
| Controls | 5 |
| Patients breast cancer | 11 |

The statistical analysis (Table 6) demonstrates the accuracy of the Calponin-h2 ELISA in differentiating breast cancer patients from healthy controls. It can thus be concluded that Calponin-h2 is a suitable marker for the prediction/detection of breast cancer and the prognosis of a patient diagnosed with of breast cancer. Also, Calponin-h2 may be used as a reliable marker for determining the efficacy of a treatment regimen of breast carcinoma in a patient.

All documents cited herein, are hereby incorporated by reference in their entirety.

The inventions illustratively described herein may suitably be practiced in the absence of any element or elements, limitation or limitations, not specifically disclosed herein. Thus, for example, the terms “comprising”, “including”, “containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the inventions embodied therein herein disclosed may be resorted to by those skilled in the art, and that such modifications and variations are considered to be within the scope of this invention. The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein. In addition, where features or aspects of the invention are described in terms of Markush groups, those skilled in the art will recognize that the invention is also thereby described in terms of any individual member or subgroup of members of the Markush group. Further embodiments of the invention will become apparent from the following claims.

1. A method of diagnosing or determining the prognosis of breast carcinoma in a patient, wherein the method comprises:
determining the level of Calponin-h2 and/or CALML5 in a sample obtained from the patient; wherein if the level of Calponin-h2 and/or CALML5 is increased relative to that of a healthy individual, said patient is diagnosed with breast carcinoma, or given an unfavorable prognosis.

2. (canceled)

3. A method of determining the efficacy of a treatment regimen of breast carcinoma in a patient, wherein the method comprises:
determining the level of Calponin-h2 and/or CALML5 in a first sample obtained from the patient before said treatment regimen has commenced and a second sample obtained from the patient during or after said treatment regimen; wherein a decrease of the level of Calponin-h2 and/or CALML5 in the second sample relative to the first sample indicates that the treatment is effective.

4. The method of claim 1, wherein the method further comprises determining the level of one or more additional markers.

5. The method of claim 4, wherein the one or more additional markers are selected from the group consisting of hsp27, estrogen receptor, progesterone receptor, EGFR, Her2, circulating DNA, circulating RNA, circulating tumor cells, upa/PAI1, miRNA, ki67, Bone Sialoprotein, CA15-3, CA27.29, CEA, P53, Cathepsin D, Cyclin E, Vitronectin, Vimentin, S100, MMP11, CTSL2, STK15, Survivin, Cyclin B1, MYBL2, GSTM1, BAG1, 1T1H4, C3a-complement, GCDFB-15, ApoD, alpha-1-acid glycoprotein, the protein in Spot C of the 2D-gel-electrophoresis (FIG. 1, 25 kDa, pI 4), the protein in Spot E of the 2D-gel-electrophoresis (FIG. 1, 20 kDa, pI 4,5), CA15-3, CA27.29, CEA, the protein in spot F of the 2D-gel-electrophoresis (FIG. 1, 20 kDa, pI 4), CEACAM1, tissue inhibitors of metalloproteinases (TIMP)-3, Calgranulin A, LyGDI, RhoA, Profilin, and Apo-C1.

6. The method of claim 1, wherein the sample is a biological sample.

7. The method of claim 6, wherein the biological sample is a body fluid, cell or tissue sample.

8. The method of claim 7, wherein the body fluid is selected from the group consisting of blood, serum, plasma, urine, nipple aspirate fluid and saliva.

9. The method of claim 1, wherein the patient is a human.

10. The method of claim 1, wherein determining the level of Calponin-h2 and/or CALML5 comprises determining the expression level of Calponin-h2 and/or CALML5.

11. The method of claim 1, wherein determining the level of Calponin-h2 and/or CALML5 comprises determining the Calponin-h2 and/or CALML5 protein and/or mRNA level in a sample.

12. The method of claim 11, wherein the protein level is determined by an immunoassay, ELISA, mass spectrometry, chromatography, Western Blot, or gel electrophoresis.

13. The method of claim 11, wherein the mRNA level is determined by PCR, gel electrophoresis, or Northern Blot.

14. A breast carcinoma diagnostic or prognostic kit, wherein the kit comprises reagents for determining the level of Calponin-h2 and/or CALML5 in a biological sample obtained from a patient.

15. (canceled)

16. The kit of claim 14, wherein the kit further comprises reagents for determining the level of one or more additional markers.

17. The kit of claim 16, wherein the one or more additional markers are selected from the group consisting of hsp27, estrogen receptor, progesterone receptor, EGFR, Her2, circulating DNA, circulating RNA, circulating tumor cells, upa/PAI1, miRNA, ki67, Bone Sialoprotein, CA15-3, CA27.29, CEA, P53, Cathepsin D, Cyclin E, Vitronectin, Vimentin, S100, MMP11, CTSL2, STK15, Survivin, Cyclin B1, MYBL2, GSTM1, BAG1, 1T1H4, C3a-complement, GCDFB-15, ApoD, alpha-1-acid glycoprotein, the protein in Spot C of the 2D-gel-electrophoresis (FIG. 1, 25 kDa, pI 4), the protein in Spot E of the 2D-gel-electrophoresis (FIG. 1, 20 kDa, pI 4,5), CA15-3, CA27.29, CEA, the protein in spot F of the 2D-gel-electrophoresis (FIG. 1, 20 kDa, pI 4), CEACAM1, tissue inhibitors of metalloproteinases (TIMP)-3, Calgranulin A, LyGDI, RhoA, Profilin, and Apo-C1.

18. The kit of claim 16, wherein the biological sample is a body fluid, cell sample or tissue sample.

19. The kit of claim 18, wherein the body fluid is selected from the group consisting of blood, serum, plasma, urine, nipple aspirate fluid and saliva.

20. The kit of claim 14 wherein the patient is a human.

21. The kit of claim 14, wherein the kit further comprises a binding partner capable of detecting the presence of the protein and/or mRNA level of Calponin-h2 and/or CALML5.

22. The kit of claim 14, wherein the kit further comprises reagents for conducting an assay selected from the group consisting of an immunoassay, ELISA, mass spectrometry, chromatography, Western Blot, gel electrophoresis, PCR, and Northern Blot.

23. The method of claim 3 wherein the patient is a human.

24. The method of claim 3 wherein the sample obtained from patient is a body fluid, cell or tissue sample.

25. The method of claim 3 wherein determining the level of Calponin-h2 and/or CALML5 comprises determining the expression level of Calponin-h2 and/or CALML5.


SEQUENCE LISTING (3 sequences)

SEQ ID NO: 1 - protein, 146 aa, Homo sapiens
MAGELTPEEEAQYKKAFSAVDTDGNGTINAQELGAALKATGKNLSEAQLRKLISEVDSDGDGEISFQEFLTAAKKARAGLEDLQVAFRAFDQDGDGHITVDELRRAMAGLGQPLPQEELDAMIREADVDQDGRVNYEEFARMLAQE

SEQ ID NO: 2 - protein, 309 aa, Homo sapiens
MSSTQFNKGPSYGLSAEVKNRLLSKYDPQKEAELRTWIEGLTGLSIGPDFQKGLKDGTILCTLMNKLQPGSVPKINRSMQNWHQLENLSNFIKAMVSYGMNPVDLFEANDLFESGNMTQVQVSLLALAGKAKTKGLQSGVDIGVKYSEKQERNFDDATMKAGQCVIGLQMGTNKCASQSGMTAYGTRRHLYDPKNHILPPMDHSTISLQMGTNKCASQVGMTAPGTRRHIYDTKLGTDKCDNSSMSLQMGYTQGANQSGQVFGLGRQIYDPKYCPQGTVADGAPSGTGDCPDPGEVPEYPPYYQEEAGY

SEQ ID NO: 3 - protein, 205 aa, Homo sapiens
MTERRVPFSLLRGPSWDPFRDWYPHSRLFDQAFGLPRLPEEWSQWLGGSSWPGYVRPLPPAAIESPAVAAPAYSRALSRQLSSGVSEIRHTADRWRVSLDVNHFAPDELTVKTKDGVVEITGKHEERQDEHGYISRCFTRKYTLPPGVDPTQVSSSLSPEGTLTVEAPMPKLATQSNEITIPVTFESRAQLGGPEAAKSDETAAK